(19) United States
(12) Patent Application Publication
(10) Pub. No.: US 2025/0314900 A1
(43) Pub. Date: Oct. 9, 2025

(54) OPTICAL SYSTEM WITH SLIM BEAM SPLITTER

(71) Applicant: Alcon Inc., Fribourg (CH)

(72) Inventor: Martin Eil, Berlin (DE)

(21) Appl. No.: 19/085,539

(22) Filed: Mar. 20, 2025

Related U.S. Application Data
(60) Provisional application No. 63/574,564, filed on Apr. 4, 2024.

(51) Int. Cl. G02B 27/14; A61B 3/13

(52) U.S. Cl. CPC G02B 27/143; A61B 3/13

(57) ABSTRACT

An optical system for viewing a sample includes oculars, a slim beam splitter positioned in an optical beam path between the oculars and the sample, and a photosensor. The slim beam splitter includes a non-cubic body having a height that extends along the optical beam path. An angled reflective surface of the slim beam splitter reflects light from the sample towards the photosensor along an outcoupled beam path and transmits light from the sample towards the oculars. The height of the non-cubic body is substantially equal to a diameter of the outcoupled beam path. A display may be configured to output a secondary light beam towards the slim beam splitter in a direction that is orthogonal to the optical beam path. The slim beam splitter may be configured to prevent light from the secondary light beam from being transmitted or reflected towards the photosensor.

### INTRODUCTION

The present disclosure relates to optical systems having a microscope, e.g., an ophthalmic microscope used by a clinician when visualizing tissue of a patient's eye under magnification. In-office ophthalmic procedures often require the clinician to illuminate and view the retina, macula, and surrounding tissue within the eye's vitreous cavity. This action is typically performed using associated eyepieces or oculars, or via a three-dimensional heads-up display. A digital camera may be used to record pixel images of the sample as needed. Visualization thus involves a dynamic and interactive examination of the patient's eye by the clinician, either with or without corresponding image collection.

A beam splitter is an optical device that is often used in conjunction with the above-noted microscope to direct reflected light from a given sample toward the oculars and camera as noted above. A beam splitter may include a partially-reflective portion and a transmissive portion, with the former sometimes having an application-suitable polarization filter or coating. Together, the portions of the beam splitter enable a predetermined amount of light entering the beam splitter to freely pass therethrough in one direction while directing the remaining portion of the incident light in another direction. The transmission-to-reflection (T/R) ratio of the beam splitter describes its relative transmission rate, e.g., with a T/R ratio of 60/40 corresponding to 60% transmission and 40% reflection.

### SUMMARY

Disclosed herein is slim-bodied optical beam splitter (“slim beam splitter”) for use as part of a microscope-equipped optical system. In accordance with the present disclosure, the slim beam splitter is characterized by its generally planar or non-cubic body. The specific proportions and construction of the slim beam splitter are thus “slim” in the sense of being of a reduced size along a direction of an optical beam path. The slim design in turn reduces the overall stack height of the microscope or other optical system incorporating the slim beam splitter, and provides other attendant benefits as set forth in detail below.

In particular, an optical system for viewing a sample is disclosed herein. An embodiment of the optical system includes oculars, a slim beam splitter positioned in an optical beam path between the oculars and the sample, and a photosensor. The slim beam splitter includes a non-cubic body having a height that extends along the optical beam path, with the slim beam splitter having an angled reflective surface. The angled reflective surface reflects light from the sample along an outcoupled beam path, which may be towards the photosensor in one or more implementations. The angled reflective surface also transmits light from the sample towards the oculars. The height of the non-cubic body is close/comparable or substantially equal to a diameter of the outcoupled beam path, e.g., slightly larger than the outcoupled beam path so as to accommodate tolerances.

An aspect of the present disclosure includes a slim beam splitter for use with oculars and a photosensor. The slim beam splitter in one or more implementations may include a non-cubic body constructed from glass, plastic, or another application suitable material, e.g., as a rectangular plate, with the body having a height along an optical beam path between a sample and the non-cubic body. An angled reflective surface is positioned within the non-cubic body. The slim beam splitter is configured to reflect a portion of light from the sample, via the angled reflective surface, towards the photosensor along an outcoupled beam path. The slim beam splitter is also configured to transmit another portion of the light from the sample towards the oculars, the height of the non-cubic body approximating a diameter of the outcoupled beam path as noted above.

Also disclosed herein is an optical system, an embodiment of which includes a microscope, oculars connected to the microscope, a slim beam splitter connected to the microscope in an optical beam path extending between the oculars and a sample, and a photosensor. The slim beam spitter includes a first partial trapezoidal body portion and a second partial trapezoidal body portion that is adhesively bonded to the first partial trapezoidal body to form a non-cubic body having height extending along the optical beam path. An angled reflective surface is disposed between the first and second partial trapezoidal bodies. A pair of glass plates may be adhered to opposing surfaces of the non-cubic body. The angled reflective surface is configured to reflect light from the sample towards the photosensor along the outcoupled beam path and transmit light from the sample towards the oculars, the height being comparable/substantially equal to a diameter of the outcoupled beam path.

The above-described and other features and advantages of the present disclosure will be apparent from the following detailed description when taken in connection with the accompanying drawings.

### BRIEF DESCRIPTION OF THE DRAWINGS

FIG. 1 is a schematic illustration of an exemplary optical system having a microscope and a slim beam splitter as disclosed herein.

FIG. 2 illustrates the slim beam splitter of FIG. 1 in accordance with a representative construction.

FIG. 3 illustrates a manufacturing sequence for constructing the slim beam splitter shown in FIG. 2.

FIG. 4 is a perspective view illustration of a portion of the slim beam splitter shown in FIGS. 1-3.

The foregoing and other features of the present disclosure are more fully apparent from the following description and appended claims when taken in conjunction with the accompanying drawings.

### DETAILED DESCRIPTION

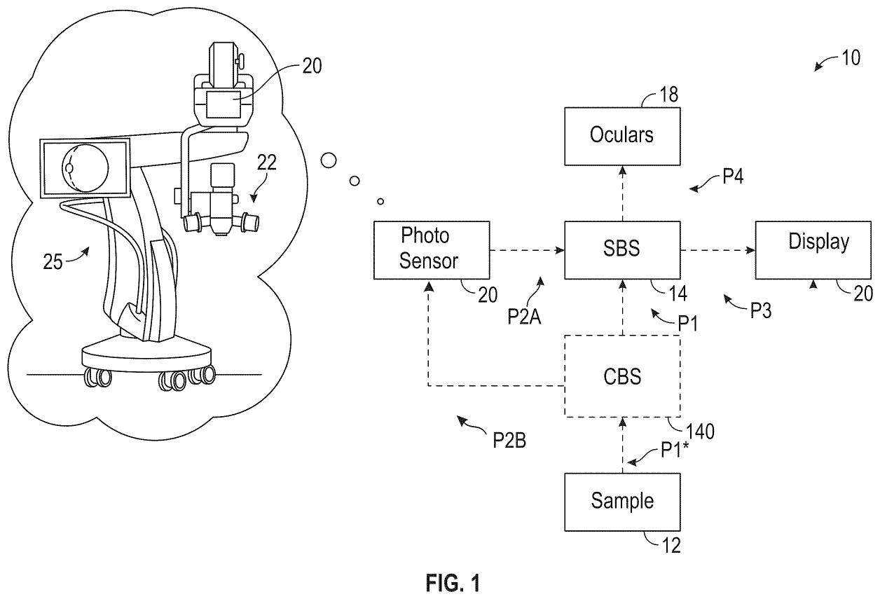

Referring to the drawings, wherein like reference numbers refer to like components throughout the various Figures, FIG. 1 illustrates an optical system 10 that an attending clinician (not shown) may use to visualize a sample 12 in real-time. In a non-limiting and representative ophthalmic use scenario, the sample 12 includes a patient's ocular tissue. However, other samples 12 of an organic or inorganic nature are possible within the scope of the disclosure. While non-limiting ophthalmic examples and use cases are provided herein for illustration, the present teachings may also be applied to a wide variety of applications in which beam splitters are typically employed.

As contemplated herein, the optical system 10 includes a slim beam splitter (SBS) 14 and a display 16, with the latter device possibly being constructed as a projector device operable for superimposing alphanumeric graphics, text, or other information on images of the sample 12 when the sample 12 is viewed through one or more eyepieces or oculars 18 of a microscope 22. The display 16 shown schematically in FIG. 1 may be variously embodied as a liquid-crystal on silicon (LCoS) projection engine, a liquid-crystal display (LCD), or an organic LED (OLED). The optical system 10 may also include a photosensor 20, e.g., a photodiode array or a camera, such as a charge-coupled device (CCD) or complementary metal-oxide semiconductor (CMOS) camera, which would allow the clinician to collect digital pixel images of the sample 12 as needed.

The slim beam splitter 14 of FIG. 1 in accordance with the present disclosure is configured to direct light from multiple light sources towards different target destinations. For example, the slim beam splitter 14 may be an integral component of the microscope 22, with the microscope 22 also possibly including or being in communication with the display 16, the oculars 18, and the photosensor 20. As shown in an inset in FIG. 1, for instance, the microscope 22 may be connected to a multi-axis surgical robot 25 and positioned relative to the sample 12 within a surgical suite.

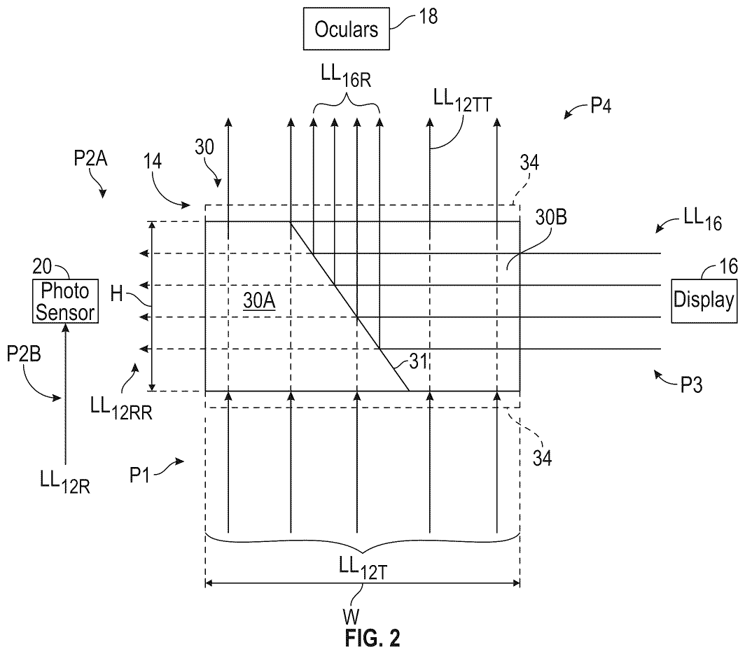

As appreciated in the art, an optical beam path (P1) is the particular path that incident light from the sample 12 will follow within the above-summarized optical system 10. In one or more implementations, an optional cube-shaped beam splitter (CBS) 140 may be used upstream of the slim beam splitter 14 to reflect light towards the photosensor 20, with the slim beam splitter 14 reflecting light from the display 16 towards the oculars 18 via the outcoupled beam path (P4). In such an embodiment, the cubic beam splitter 140 may be positioned between the sample 12 and the slim beam splitter 14, in which case the optical beam path P1 is coaxial with and downstream of an optical beam path P1* from the sample 12. The slim beam splitter 14 and the cubic beam splitter 140 may be used in concert in some applications in which the photosensor 20 has a relatively large aperture. For instance, beam path P2B of FIG. 2 would coincide with the large aperture. Image injection from the display 16 may be performed solely using the slim beam splitter 14 in such an embodiment, with the slim beam splitter 14 being interposed between the cubic beam splitter 140 and the oculars 18. The cubic beam splitter 140 may be omitted in other constructions, provided incident light from the display 16 is prevented from passing towards the photosensor 20, e.g., via use of a polarization coating on the slim beam splitter 14 or other suitable techniques.

Incident light from the display 16 is transmitted to the slim beam spitter 14 along an input beam path (P3). As described in detail below with reference to FIG. 2, the slim beam splitter 14 is configured to transmit light from the sample 12 to the oculars 18 along an outcoupled beam path (P4). When used, the optional cubic beam splitter 140 may reflect light from the sample 12 towards the photosensor 20 along another outcoupled beam path (P2B), with light also reflected by the slim beam spitter 14 to the photosensor 20 via an outcoupled beam path (P2A). In a configuration using a cubic beam splitter 140, light from the display 16 is not transmitted to the photosensor 20 so as not to see anything of the display 16 via the photosensor 20, e.g., so as not to confuse image processing algorithms. However, other applications may benefit from seeing the display 16 via the photosensor 20. Thus, the optical system 10 as illustrated schematically in FIG. 1 uses the slim beam splitter 14 of the present disclosure in a particular manner to transmit and reflect light from the sample 12 and display 16 during real-time visualization and examination of the sample 12.

In optical systems of types frequently used within a modern ophthalmic suite, the optical beam path (P1) is much larger than the outcoupled beam path (P2A) to the photosensor 20, often by a factor of 1.25× to 30×. To that end, a cube-shaped beam splitter like the optional cubic beam spitter 140 of FIG. 1 is customarily sized to match the much larger optical beam path (P1). For example, glass cubes having side lengths of 20 millimeter (mm) for an exemplary 20 mm optical beam path (P1) are common across a wide range of optical applications. In most cases, one would use a slim beam splitter 14 that is slightly larger than the beam path, e.g., 20 mm for a beam path of about 18-19 mm. However, it is recognized herein that integration of a cubic or quadratic beam splitter into a modern microscope stack greatly increases the resulting stack height. Cube-shaped beam splitters may also decrease the working distance between the sample 12 and internal lenses of the oculars 18 of the microscope 22. Cube-shaped beam splitters therefore remain suboptimal when used in certain microscope-based applications.

In contrast, the slim beam splitter 14 described in detail herein purposefully matches the smaller size of the outcoupled beam path (P2A). This may be only about 2 mm, or about 10% or less of the width of the above-noted 20 mm diameter example of the optical beam path (P1). The height of the slim beam splitter 14 may be varied to match an aperture size of the outcoupled beam path (P2A). For instance, if the outcoupled beam path (P2A) has a diameter of 6 mm, the height of the slim beam splitter 14 may also be about 6 mm. The slim beam splitter 14 of the present disclosure thus contributes minimally to overall stack height of the microscope 22 relative to cubic designs, while at the same time benefitting vignetting (which increases with stack height) while minimizing losses in the transmission direction. The reduced working distance may also increase clinician comfort level and increase the field-of-view relative to standard cube-shaped/quadratic beam splitters.

Referring now to FIG. 2, when viewing the sample 12 shown schematically in FIG. 1, the slim beam splitter 14 is operable for receiving a primary light beam (LL12T) from the sample 12 along the optical beam path (P1), with the primary light beam (LL12T) being emitted from and/or reflected by the sample 12 when the sample 12 is illuminated. Illumination of the sample 12 may be achieved using a light source (not shown) connected to the microscope 22, via internal lighting such as endo-illuminators or chandeliers, or via an external light source.

The display 16 may output a secondary light beam (LL16) along input beam path (P3), e.g., in an orthogonal direction relative to the optical beam path (P1). In such a setup, the slim beam splitter 14 also receives the secondary light beam (LL16) from the display 16. Reflected portions of the primary light beam (LL12T) and the secondary light beam (LL16) are then directed by the slim beam splitter 14 towards the oculars 18 and the photosensor 20 as reflected light (LL12RR) and (LL16R), respectively. This occurs along the respective outcoupled beam paths (P4) and (P2A). In operation, therefore, nearly all of the incident light from the display 16, i.e., the secondary light beam (LL16), will pass through the reflective portion/reflective surface 31 of the slim beam splitter 14. In contrast, most of the primary light beam (LL12T) will pass through areas of the slim beam splitter 14 lacking such reflective material 31. In effect, the oculars 18 will receive as much as or more light of a similarly-sized cubic reflector, while still receiving display data from the display 16 when such data is overlayed onto an image viewed by the clinician via the oculars 18.

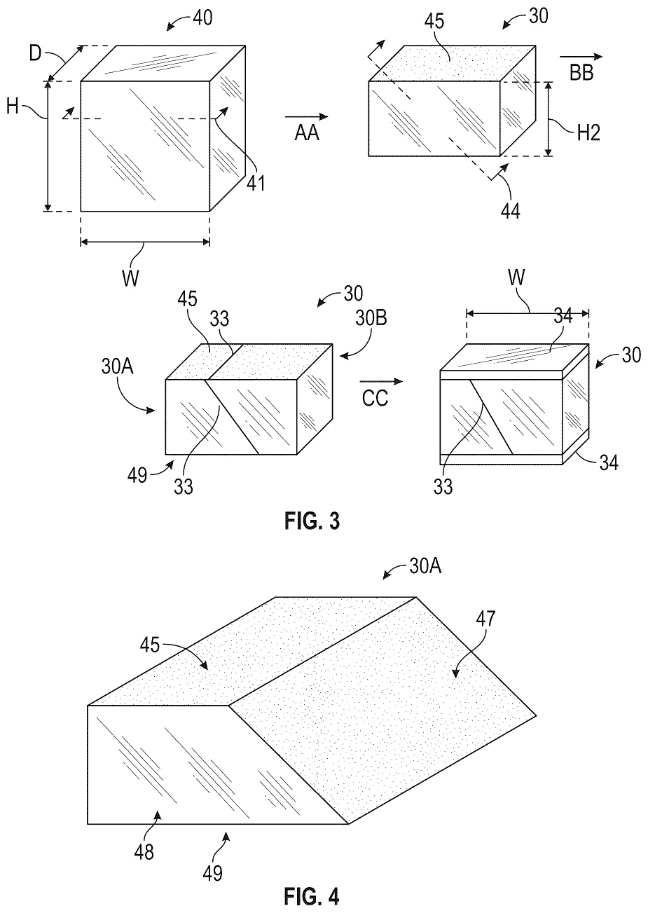

The slim beam spitter 14 of FIG. 2 includes a body 30 having a height (H) along the optical beam path (P1). As described below with reference to FIGS. 3 and 4, the body 30 may be constructed from joined first and second partial trapezoidal body portions 30A and 30B and optional glass plates 34 on opposing surfaces of the body 30. The body 30 has a size that is substantially equal to that of the outcoupled beam path (P2A), i.e., within ±10-15% or within ±1-5%. The slim beam splitter 14, which contains therein an angled reflective surface 31, is thus configured to transmit a first portion of the primary light beam (LL12T) along the outcoupled beam path (P4) towards the oculars 18 as transmitted light beam (LL12TT). Additionally, the slim beam splitter 14 is configured to reflect a second portion of the primary light beam (LL12T) via the angled reflective surface 31 along the output coupled path (P2A) towards the photosensor 20. Such reflected light (LL_{12RR}) is thereby directed to and detectable by the photosensor 20. When using the cubic beam splitter 140 of FIG. 1, a reflected light beam (LL12R) may be directed towards the photosensor 20 along the outcoupled beam path (P2B) as noted above.

A volume or envelope defined by the slim beam splitter 14 is generally planar or flat. To that end, the body 30 may be plate-like, with a width (W) and the height (H). When positioned in the optical beam path (P1), the width (W) of the body 30 is arranged normal or perpendicular to the optical beam path (P1). In a non-limiting exemplary construction, the height (H) of the body 30 may be less than about 6-8 mm, with as little as about 2-4 mm being possible for certain applications. The width (W) may be less than about 18-22 mm in such an embodiment, with other possible sizes being possible depending on the desired size of the outcoupled beam path (P2A).

When a beam splitter of any type is included in a microscope stack, e.g., of the microscope 22 of FIG. 1, the resultant stack height is increased. A standard beam splitter having a cubic volume as noted above ensures that, if a small light beam is coupled out of or into the system, the stack height will grow by at least the size of the optical beam path (P1). In contrast, the slim beam splitter 14 of FIG. 2 is sized to the much smaller outcoupled beam (P2A). Thus, light is lost in the transmission direction at a lower rate, which in turn improves the transmission/reflection (T/R) rate from the perspective of the clinician.

Referring now to FIG. 3, the slim beam splitter 14 of FIGS. 1 and 2 may be constructed in one or more embodiments from a monolithic cubic or rectangular block 40 of an optically-suitable glass, plastic, quartz, or another material, for instance using an abrasive cutting tool or a laser. The height (H), depth (D), and width (W) of the block 40 may be equal as a starting point, or the original height (H) may differ from the depth (D) and width (W) depending on the desired dimensions of the body 30. Continuing with this process as indicated by arrow AA, the body 30 of the slim beam splitter 14 of FIGS. 1 and 2 is then separated from the block 40, for instance by severing a portion of the block 40 along a cut line 41. This in turn would provide the body 30 with a reduced height (H2) relative to the original height (H) of the block 40. An exposed/rough upper surface 45 and possibly a rough lower surface 49 may result from such a cutting process.

Proceeding as indicated by arrow BB, the body 30 may be divided along an angled cut line 44 into the respective first and second partial trapezoidal body portions 30A and 30B. As shown in FIG. 4, the first partial trapezoidal body portion 30A may have the upper surface 45, side surfaces 48 (one of which is visible from the perspective of FIG. 4), the lower surface 49, and an angled surface 47. Of these, the upper surface 45, the angled surface 47, and possibly the lower surface 49 may be relatively rough or unfinished after completing the above-noted cutting process, i.e., presenting more pronounced surface asperities relative to the smooth surface finish of the side surfaces 48.

After depositing, applying, or otherwise coating the angled surface 47 of the first partial trapezoidal body portion 30A of FIG. 3 (and/or the substantially-identical second partial trapezoidal body portion 30B of FIG. 2) with reflective material, the respective first and second trapezoidal body portions 30A and 30B are then bonded together with an optically transmissive adhesive to reform the body 30. An interface 33 is thus formed along mating surfaces of the first and second partial trapezoidal body portions 30A and 30B.

Proceeding as indicated by arrow CC, the upper and lower surfaces 45 and 49 may remain relatively rough after cutting or dividing as noted above. Desired optical transmission characteristics may therefore be restored in one or more embodiments by adhering glass plates 34 to opposing surfaces of the body 30, i.e., the upper and lower surfaces 48 and 49, using an optically suitable and materially compatible adhesive. The glass plates 34 when adhered or bonded in this manner are parallel to upper and lower surfaces 48 and 49 and perpendicular to the side surfaces 48.

While the slim beam splitter 14 described herein may be configured with a transmission-to-reflection (T/R) ratio or “splitting ratio” of anywhere between 1/99 and 99/1, the slim beam splitter 14 in a particular application may have a T/R ratio of at least 50/50 (with polarization coating or splitter), 70/30, or in a range of about 90/10 to about 95/5 in other implementations. The ratio is from the center of the slim beam splitter 14 on the angled surface 47 where the reflective surface 31 resides (i.e., for the 50/50 example, the total transmission would exceed 50% when one considers the beam splitter 14 as a whole). The desired T/R ratio may be achieved using a polarization coating on the angled surface 47 or along the interface 33 in one or more embodiments, e.g., to prevent light from the display 16 from being transmitted or reflected towards the photosensor 20. A substantial majority of incident light that passes into the slim beam splitter 14 is therefore transmitted through the slim beam splitter 14 rather than being reflected from the angled reflective surface 31 of FIG. 2.

In yet another possible construction, the angled reflective surface 31 of FIG. 2 may be integrally formed within the body 30 without first cutting or otherwise separating the body 30 into the first and second partial trapezoidal body portions 30A and 30B of FIGS. 2 and 3. In other words, the body 30 may remain in the form of a monolithic planar piece or plate as shown in FIG. 2. Formation of the body 30 in this manner may entail the deposition or insertion of a reflective material at a predetermined angle within the body 30 as the glass, plastic, or other materials of the body 30 cool and solidify during manufacturing.

The above solutions of FIGS. 1-4 depart from the art of cubic beam splitters to reduce stack height in the microscope 22 of FIG. 1. The representative approach of FIGS. 2 and 3 thus enables construction of substantially flat or plate-like/non-cubic and therefore slim beam splitter 14, possibly with the added glass plates 34 when cutting processes leave rough or high-asperity surfaces. Other techniques may be used, e.g., repolishing, especially at the edge where parts of the body 30 come together within the optical beam path (P1).

By sizing the height (H) of the body 30 to the required size of the outcoupled beam path (P2A), e.g., a 1:1 relationship, the overall stack height is reduced and less light is wasted in the transmission direction, i.e., between the sample 12 and the oculars 18. The T/R rate is thereby improved from the perspective of the clinician using the oculars 18 of FIG. 1. These and other attendant benefits will be appreciated by those skilled in the art in view of the foregoing disclosure.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

We claim:

1. An optical system for viewing a sample, comprising:
oculars;
a slim beam splitter positioned in an optical beam path between the oculars and the sample, the slim beam splitter including:
a non-cubic body having a height along the optical beam path; and
an angled reflective surface; and
a photosensor, the angled reflective surface being configured to (i) reflect light from the sample towards the photosensor along an outcoupled beam path, and (ii) transmit light from the sample towards the oculars, the height of the non-cubic body being substantially equal to a diameter of the outcoupled beam path.

2. The optical system of claim 1, wherein the height of the non-cubic body is less than about 8 millimeters.

3. The optical system of claim 2, wherein the height of the non-cubic body is about 2 millimeters.

4. The optical system of claim 1, wherein the slim beam splitter includes a pair of glass plates, and wherein each respective glass plate of the pair of glass plates is adhered to opposing surfaces of the non-cubic body.

5. The optical system of claim 1, wherein the slim beam splitter has a transmission-to-reflection ratio of about 50/50 to about 99/1.

6. The optical system of claim 5, wherein the transmission-to-reflection ratio is about 90/10 to about 95/5.

7. The optical system of claim 1, further comprising:
a microscope, wherein the slim beam splitter is a component of the microscope.

8. The optical system of claim 1, further comprising:
a display configured to output a secondary light beam towards the slim beam splitter in a direction that is orthogonal to the optical beam path, wherein the slim beam splitter is configured to prevent light from the secondary light beam from being transmitted or reflected towards the photosensor.

9. The optical system of claim 1, wherein the body of the slim beam splitter includes a first partial trapezoidal body portion and a second partial trapezoidal body portion, and wherein the first partial trapezoidal body portion and the second partial trapezoidal are adhesively bonded together.

10. The optical system of claim 1, wherein the non-cubic body of the slim beam splitter is a rectangular plate constructed of glass or plastic.

11. A slim beam splitter for use with oculars and a photosensor, comprising:
a non-cubic body constructed from glass, wherein the non-cubic body has height that extends along an optical beam path between a sample and the non-cubic body; and
an angled reflective surface positioned within the non-cubic body, wherein the slim beam splitter is configured to:
reflect a portion of light from the sample, via the angled reflective surface, towards the photosensor along an outcoupled beam path; and
transmit another portion of the light from the sample towards the oculars, the height of the non-cubic body being substantially equal to a diameter of the outcoupled beam path.

12. The slim beam splitter of claim 11, wherein the height of the non-cubic body is less than about 8 millimeters.

13. The slim beam splitter of claim 12, wherein the height of the non-cubic body is less than about 2 millimeters.

14. The slim beam splitter of claim 11, further comprising:
a pair of glass plates adhered to opposing surfaces of the non-cubic body.

15. The slim beam splitter of claim 11, wherein the slim beam splitter has a transmission-to-reflection ratio of about 50/50 to about 99/1.

16. The slim beam splitter of claim 15, wherein the transmission-to-reflection ratio is about 90/10 to about 95/5.

17. The slim beam splitter of claim 11, wherein the of the non-cubic body of the slim beam splitter includes a first partial trapezoidal body portion and a second partial trapezoidal body portion, the first partial trapezoidal body portion and a second partial trapezoidal body portion being adhesively bonded together.

18. An optical system, comprising:
a microscope;
oculars connected to the microscope;
a slim beam splitter connected to the microscope in an optical beam path extending between the oculars and a sample, the slim beam spitter including:
a first partial trapezoidal body portion;
a second partial trapezoidal body portion that is adhesively bonded to the first partial trapezoidal body to form a non-cubic body having height along the optical beam path;
an angled reflective surface disposed between the first partial trapezoidal body and the second partial trapezoidal body; and
a pair of glass plates, each respective one of which is adhered to opposing surfaces of the non-cubic body; and
a photosensor, the angled reflective surface being configured to reflect light from the sample towards the photosensor along an outcoupled beam path and transmit light from the sample towards the oculars, the height being substantially equal to a diameter of the outcoupled beam path.

19. The optical system of claim 18, further comprising:
a display configured to output a secondary light beam towards the slim beam splitter in a direction that is orthogonal to the optical beam path, wherein the slim beam splitter is configured to prevent light from the secondary light beam from being transmitted or reflected towards the photosensor.

20. The optical system of claim 18, wherein the height of the non-cubic body is less than about 2-4 millimeters, and wherein a transmission/reflection ratio of the slim beam splitter is about 95/5 to about 99/1.
